(19) United States
(12) Patent Application Publication
(10) Pub. No.: US 2019/0282125 A1
(43) Pub. Date: Sep. 19, 2019

(54) RESPIRATION FROM A PHOTOPLETHYSMOGRAM (PPG) USING FIXED AND ADAPTIVE FILTERING

(71) Applicant: Nonin Medical, Inc., Plymouth, MN (US)

(72) Inventor: James E. Durnin, Plymouth, MN (US)

(21) Appl. No.: 15/922,064

(22) Filed: Mar. 15, 2018

(51) Int. Cl. A61B 5/08; A61B 5/00; A61B 5/024; A61B 5/1455; G16H 50/20

(52) U.S. Cl. CPC A61B 5/0806; A61B 5/0816; G16H 50/20; A61B 5/02416; A61B 5/14551; A61B 5/725

(57) ABSTRACT

Methods and systems for determining a respiration rate (RR) of a subject are disclosed. In one embodiment, a method includes sampling a PPG signal at a first frequency, filtering the PPG signal with a first high-pass filter, receiving an output from the first high-pass filter and filtering the output at a second frequency in a second high-pass filter, counting positive- and negative-edge pulses of a portion of the PPG signal to determine breath-time intervals caused by an influence of the respiration rate on the PPG signal, and determining an average of the breath-time intervals for the positive-edge zero-crossings and the negative-edge zero-crossings to derive an estimate of the RR. In other embodiments, a central frequency of components of the PPG signal is determined based on bandpass filters and a feedback mechanism to estimate β and select an appropriate adaptive filter to determine the RR. Other methods and systems are disclosed.

### TECHNICAL FIELD

The inventive subject matter disclosed herein relates to deriving respiration rates of a subject (e.g., a human patient) from optically-based physiological sensor devices, such as a pulse oximeter, that produce an output in the form of a photoplethysmogram (PPG).

### BACKGROUND

A wide range of devices exist that depend upon the transmission of optical signals to monitor or measure various biological or environmental parameters of a patient. For example, various forms of blood oximetry devices employ the transmission and reception of signals in the measurement of one or more biological or environmental parameters of a patient.

Blood oximetry devices, or pulse oximeters, are commonly used to monitor or measure oxygen saturation levels of blood in a body organ or tissues, including blood vessels, or the oxidative metabolism of tissues or organs. An example of an optical oximeter is disclosed in U.S. Pat. No. Re 33,643, entitled “Single Channel Pulse Oximeter.” Pulse oximetry is a technology used to measure the oxygen level in a subject's blood as well as the subject's heart rate. A finger pulse oximeter is equipped with technology to rapidly detect changes in the subject's blood oxygen level. These devices are also often capable of and are used to determine pulse rate and volume of blood flow in organs or tissues, or to monitor or measure other biological or environmental parameters.

A blood oximetry device measures the levels of the components of one or more signals of one or more frequencies as transmitted through or reflected from tissue or an organ to determine one or more biological or environmental parameters, such as blood oxygenation level and blood volume or pulse rate of a patient.

Additionally, respiration affects cardiac cycles by varying the intrathoracic pressure within the pleural cavity of an animal (e.g., a human) subject. The intrathoracic pressure is the pressure between the thoracic wall and the lungs. Since the heart resides in the thoracic cavity between the lungs, the partial pressure due to inhalation and exhalation during breathing influences the pressure on the venae cavae. Therefore, since respiration affects the cardiac cycle, the PPG contains signal components caused by the respiratory cycles of inhaled and exhaled breaths. Consequently, the PPG signal contains information that may be extracted to determine the subject's respiration rate in breaths per minute (BPM).

Blood oximetry devices may also be constructed as directly connected devices, that is, devices that are connected directly to a patient and that directly present the desired information or directly record the information, and as remote devices, that is, devices attached to a patient and transmitting the measurements to a remote display, monitoring or data collection device.

Blood oximetry devices measure blood oxygen levels, pulse rate, and volume of blood flow by emitting radiation in a frequency range, such as the red or near infrared range, wherein the transmission of the radiation through or reflectance of the radiation from the tissues or organ is measurably affected by the oxygen saturation levels and volume of the blood in the tissues or organ. A measurement of the signal level transmitted through a tissue or organ or reflected from a tissue or organ may then provide a measurement or indication of the oxygen saturation level in the tissue or organ. The transmitted or reflected signals may be of different frequencies which are typically affected in measurably different ways or amounts by various parameters or factors or components of the blood.

Parameters represented by transmitted or reflected signals may be represented by different and related or unrelated parameters of the received signals. For example, a signal transmitted through or reflected from tissue or an organ to measure, for example, blood oxygenation or flow, may have a constant or “DC” component due to the steady state volume of blood in the tissue or organ and time varying or “AC” components indicative of the time varying volume of blood flowing through the tissue or organ due to the heart beat of the body. Each signal component may provide different information, and may provide information that may be used together to generate or determine further information. What is needed is a way to determine quickly and accurately the respiration rate of a subject (e.g., a human patient) using date from the PPG.

### BRIEF DESCRIPTION OF THE FIGURES

FIG. 1A shows an unmodulated signal of a PPG of a cardiac pulse;

FIGS. 1B through 1D show various modulations of the PPG of FIG. 1A due to respiration through two complete respiratory cycles;

FIGS. 2A-2C show front-end processing methods for each of the three fundamental signals (DC, pT, and pM);

FIG. 3 shows a fixed-filter algorithm for a preliminary determination of respiration from a frequency-modulated signal;

FIG. 4 shows a running DC average signal obtained over two single-pulse lengths;

FIG. 5A shows a plot of the intensity of a signal value, H(β), as a function of β for each of β bandpass filters;

FIG. 5B shows normalized intensity plots for outputs from each of the β linear-phase bandpass filters, starting at β=0, for each of 29 taps in accordance with an adaptive-filtering embodiment of the disclosed subject matter;

FIGS. 6A-6C show additional operations for determining a respiration rate using the adaptive-filter algorithm for each of the three fundamental signals;

FIGS. 7A and 7B show example graphs used in a testing protocol for spectral calibration of the adaptive filters described herein;

FIGS. 8A through 8D show separate ones of the spectra for each of the fundamental signals, plus the average of the three signals as discussed with reference to FIGS. 6B and 6C, plotted against a true value of β;

FIGS. 9A and 9B show an impact of spectral equalization prior to averaging three fundamental spectra as show with reference to FIGS. 8A through 8C;

FIG. 10A provides additional details on increasing the accuracy of β as determined by the adaptive-filter algorithm based on using a number of inputs to develop a second-order surface response function and a resulting transfer function estimate of β;

FIG. 10B shows combining spectral and time-domain estimates of β to produce a nonlinear enhancement of resolution of an actual value of β;

FIGS. 11A-11C show waveform examples with dynamic 1 estimates in pulse time; and

FIG. 12 shows a simplified block diagram of a machine in an exemplary form of a computing system within which a set of instructions, for causing the machine to perform any one or more of the methodologies discussed herein, may be executed.

### DETAILED DESCRIPTION

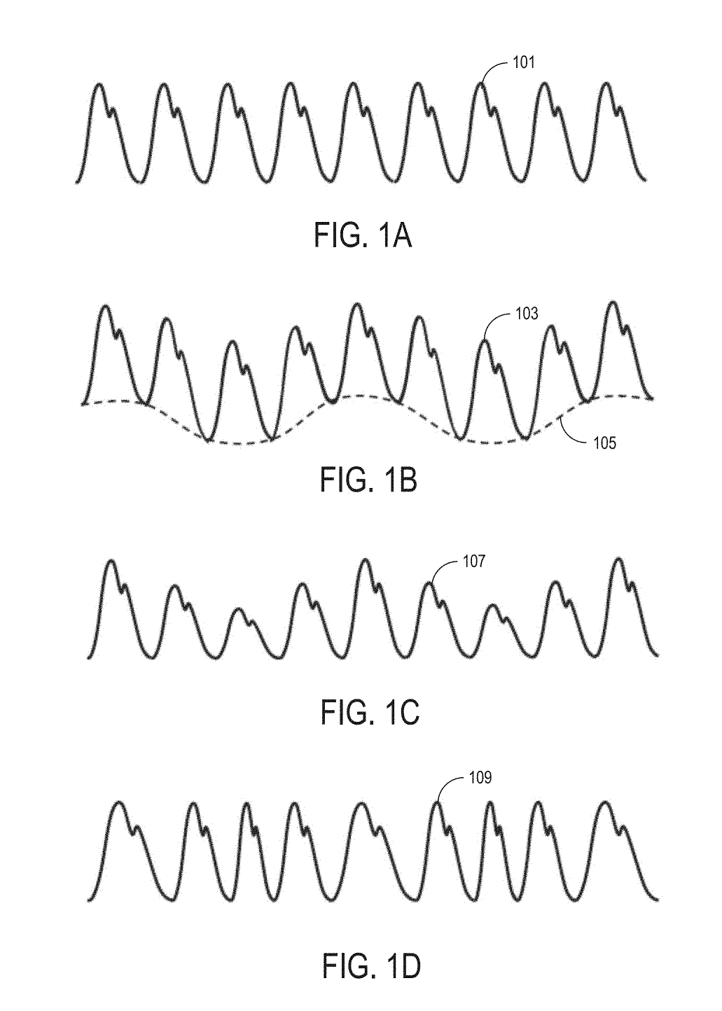

As discussed above, changes in intrathoracic pressure during respiration cycles cause modulations to a PPG signal. In FIGS. 1A through 1D, various signals sampled from a pulse oximeter coupled to a subject are shown. In FIG. 1A, a PPG waveform 101 is shown as an unmodulated cardiac pulse. The PPG waveform 101 is an expected response signal of a cardiac pulse from a subject under test with no influence from the subject's respiration (e.g., breath rate). The PPG waveform 101 is continually repeating for a constant heart rate.

Referring now to FIGS. 1B through 1D, various modulations of the PPG of FIG. 1A due to respiration are shown through two complete respiratory cycles. The modulated PPG waveforms of FIGS. 1B through 1D depict what is occurring due to changes in blood volume in a subject's finger. The modulated PPG waveforms therefore represent the three fundamental signals referred to herein. The three fundamental signals are processed in accordance with various techniques as described in detail below.

For example, FIG. 1B shows a DC-modulated waveform 103 of the PPG modulated by an underlying baseline waveform 105. The DC modulation of the PPG is caused by a variation in venous return of blood to the heart. The DC-modulated waveform 103 may alternatively be referred to herein as the DC signal. A person of ordinary skill in the art will also recognize that, even without breathing, there would still be a DC modulation of the PPG due to Mayer waves. Mayer waves are cyclic changes in arterial blood pressure brought about by various receptors in blood vessels that relay blood pressure information to the brain in order to maintain a proper blood pressure. The Mayer waves have a frequency of about 0.1 Hz (e.g., a period of about 10 seconds). This low-frequency “noise” caused by Mayer waves is one of the signals that must be reduced or eliminated by digital or analog high-pass filtering (or a combination of both) in order to extract the actual respiration rate (RR) from a PPG waveform.

FIG. 1C shows an amplitude-modulated waveform 107 of the cardiac pulses as modulated by each of the two respiratory cycles. Changes in the pulse amplitude are caused by a variation in stroke volume and are referred to herein as a p-max or pM signal.

FIG. 1D shows a frequency-modulated cardiac pulse waveform 109 that is modulated by changes in the pulse time. Therefore, the pulse length of the PPG changes in accordance with pulse time (the variation in heart rate due to respiration). The pulse time typically increases during inspiration and decreases during respiration. The variation in heart rate is known in the art as Respiratory Sinus Arrhyrthmia (RSA) and is regulated by the Vagus Nerve. The Vagus Nerve interfaces with a portion of the autonomic nervous system for control of the heart, lungs, and digestive tract of a subject. Therefore, changes in the frequency modulation due to the pulse time are referred to herein as a pulse-time or pT signal.

All three of these fundamental signals, DC, pM, and pT, are used substantially concurrently to extract an actual respiration rate of a subject (e.g., a patient). The signal-to-noise ratio (SNR) of these three fundamental signals can vary greatly from one subject to another. For example, some subjects may have a high SNR for all three signals. For other subjects, only one of the three signals may have an SNR that is sufficiently high to extract a respiratory rate. For a small percentage of the population, none of the three signals has a high SNR. Therefore, by considering each of the three fundamental signals, a true respiration rate can be extracted for all or nearly all subjects.

Various embodiments of the inventive subject matter presented herein consider zero-crossings of the signals in a time domain. As discussed in detail below, the SNR of each of the three fundamental signals is increased or maximized by using an adaptive filter that is tuned to the time-dependent signal. Consequently, a determination is made as to the approximate frequency of the signal. The signal is then passed through a filter that is closely matched, in time, to the signal. By matching the filter width, in time, to the signal, the SNR is increased or maximized. For example, if the sampling window of the filter is too wide, extra noise is introduced. If the width is too small, the signal cannot be resolved in time.

A key parameter used in extraction of the respiration rate is beta (β). β is defined as the breath frequency when sampling at the pulse rate and is given by the following equation:

$$β = \frac{respiration rate}{pulse rate} \qquad ( 1 )$$

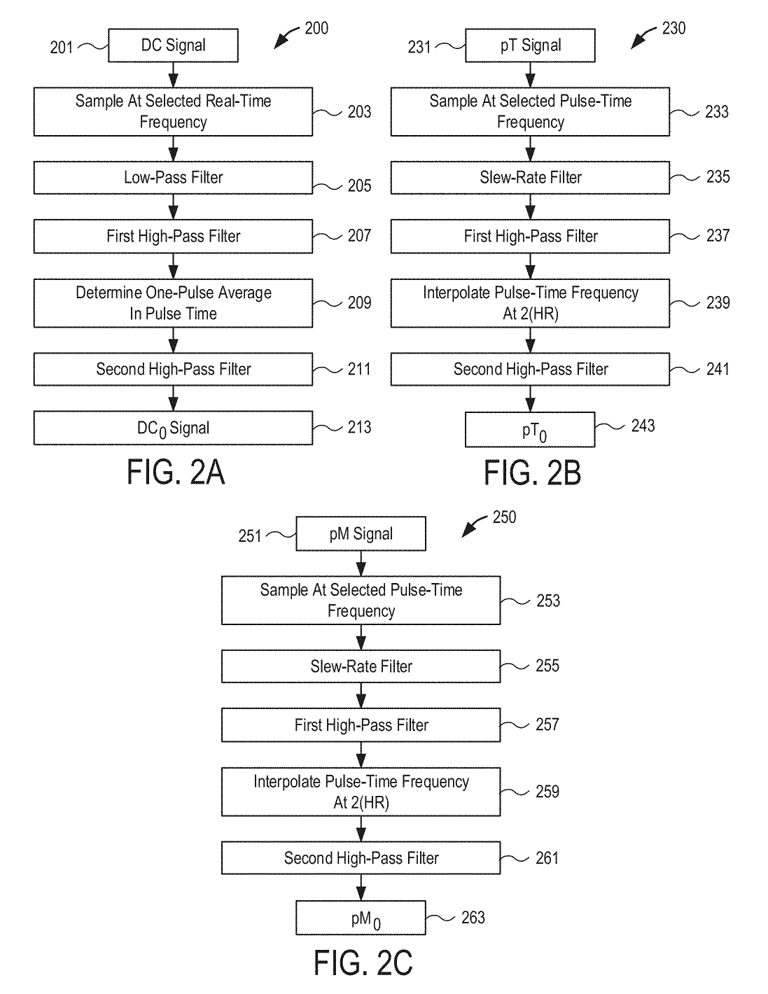

Therefore, as shown by equation (1), β is the frequency of the respiration rate in pulse time (as opposed to real time). After initial operations of front-end processing of the three signals, described below with reference to FIGS. 2A through 2C, all remaining filtering described herein is performed in pulse time (i.e., tied to the pulse rate of the subject).

Since the fundamental signals are sampled discretely, as opposed to continuously, the Nyquist sampling criteria applies. As is known to a skilled artisan, the Nyquist frequency is half the sampling frequency of any discrete signal processing system, and signal aliasing will occur at frequencies higher than the Nyquist frequency. A final sampling rate for each of the three fundamental signals (pT, pM, and DC) as described herein is two times (2×) the heart rate, which means that theoretically information content can be captures up to a value of β=1.0. However, because the information content carried by each of the three fundamental signals is inherently equivalent to sampling at only one times (1×) the heart rate, the effective Nyquist frequency (above which aliasing occurs) is β=0.5, and a respiration rate greater than half the heart rate cannot be measured. For spontaneous breathing in human subjects, the heart rate is typically four times (4×) to five times (5×) the respiration rate, and being limited to detecting respiration rates less than half the heart rate is not a significant limitation in practice.

With reference now to FIGS. 2A through 2C, front-end processing methods for each of the three fundamental signals (DC, pT, and pM) is shown.

In FIG. 2A, a DC digital-signal filtering method 200 begins at 201, where the DC signal is sampled at a selected real-time frequency at 203. In one embodiment, the sampling frequency is 75 Hz. In this embodiment, the 75 Hz sampling frequency was selected to conform with standard pulse oximetry devices currently available on the market. One purpose for using a 75 Hz sampling frequency is to provide a high resolution of the PPG waveform features. In particular, an initial inrush is recorded when a pulse oximeter is coupled to a subject (e.g., the oximeter is coupled to the subject's finger or ear). The inrush typically lasts about 100 milliseconds and defines a region where the PPG is varying most rapidly. Regardless, the skilled artisan will understand that many other sampling frequencies, including both higher and lower frequencies, may be used.

At 205 a low-pass filter eliminates much of the high-frequency signal due to the cardiac pulses and passes primarily the low-frequency signal caused by the respiration of the subject.

At 207, the signal received from 205 is passed through a first high-pass filter. In an embodiment, the first high-pass filter may have an exponential-type averaging function to provide a smoothing of the input data. Such high-pass filter types are known in the art (e.g., such as a DC blocker). This embodiment may also use the high-pass filter with a p-value of 0.00.

With regard to p-values, for a given digital signal X[n], where n is the sample number, a high-pass filtered value, D[n], (commonly referred to as a DC blocker), used in various embodiments described herein, can be categorized by a p-value as given by the mathematical equation:

D[n]=X[n]−X[n−1]+p*D[n−1]

where the parameter p satisfies the condition: 0≤p<1.

A corresponding Z-domain transfer function H(Z) is then given by:

H(Z)=D(Z)/X(Z)=[1−(Z−1)]/[1−p*(Z−1)]

which has a Zero located at Z=1 (DC), and a pole located at Z=p.

Additional determinations for p-values are described in more detail, below. If digital-filtering techniques are employed, the skilled artisan will recognize that various types of techniques may be used to smooth the data for each of the filtering steps described herein. For example, higher-degree polynomial fits, z-transfer functions, pulse transfer functions, moving average functions, and so on are known in the art.

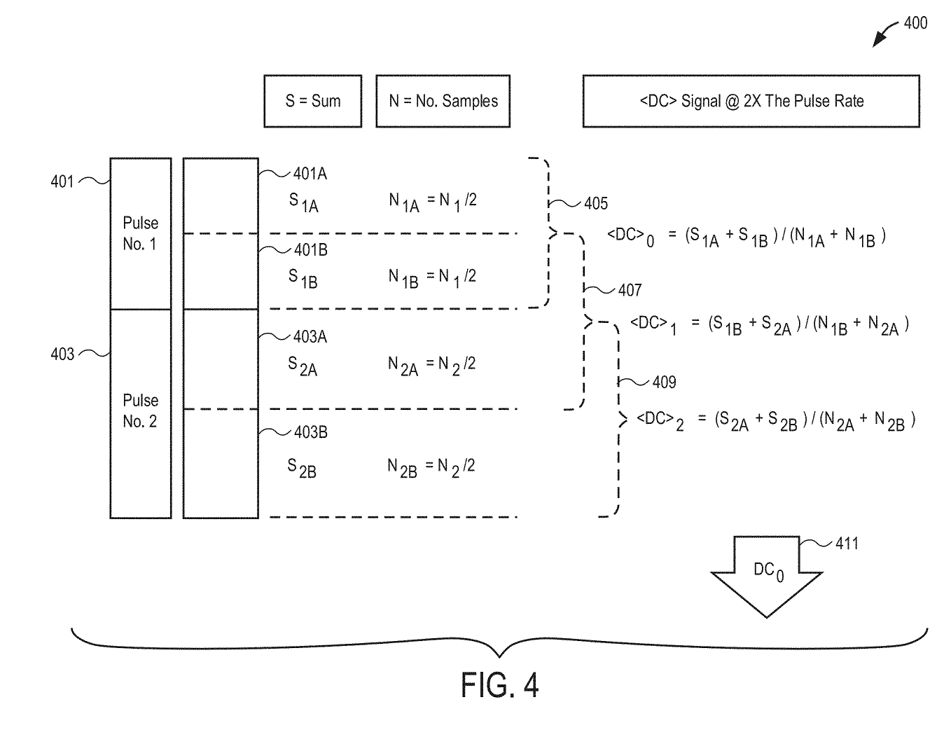

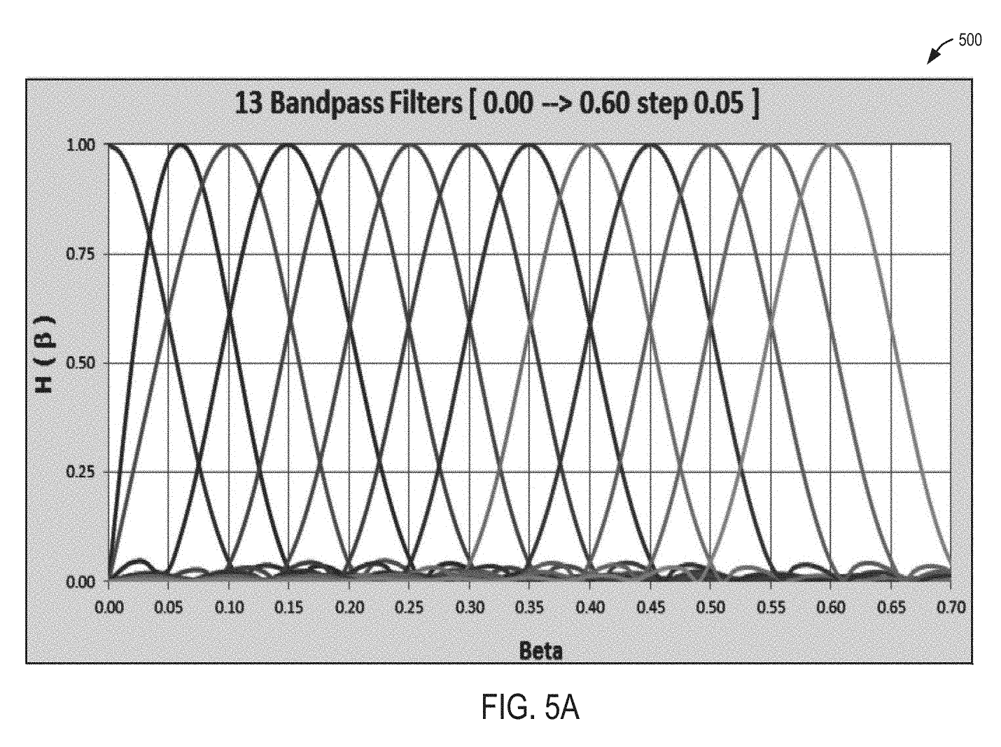

At 209, the output of the first high-pass filter is averaged over one pulse at a time. In this embodiment, the pulse is shifted by one-half pulse at a time over a sampling pulse-time frequency of two heart rate (HR) pulses. The pulse shifting technique is described in more detail with reference to FIG. 4, below.

At 211, the resulting signal output from 209 is passed through a second high-pass filter having, for example, a p-value of 0.50. A front-end processed signal of DC₀ is output at 213 from the DC digital-signal filtering method 200. In various embodiments, all but the p=0 value used in the DC signal can be determined empirically to increase or maximize SNR across the subject population. The p=0 value for the DC, when combined with averaging over one pulse, has a very special property that it produces a transfer function which depends only on β, and not on actual frequency.

The two high-pass filtering steps help reduce or eliminate frequencies due to Mayer waves, discussed above with reference to FIG. 1B, and are selectively chosen to pass frequencies related to respiration rates. Each of the high-pass filters may employ a different type of averaging function or averaging functions of the same type with different values.

In FIG. 2B, a pT signal-filtering method 230 is shown. In FIG. 2C, a pM signal-filtering method 250 is shown. Each of the pT and the pM signals are inherently determined at one-times the heart rate (1×HR). That is, the pT and the pM signals are inherently 1×HR in the sense that only one value of the pT and pM signals can be acquired from each pulse—no higher frequency of information on the pT and pM signals is possible.

With concurrent reference to FIGS. 2B and 2C, each of the pT signal and the pM signal processing begins at 231, 251, respectively, where the respective signals are sampled at a selected frequency pulse-time frequency. In an embodiment, the frequency is selected to be equivalent to the heart rate (Fₛ=1×HR). The heart rate is readily determined from the composite pulse oximeter signal and may be sampled at a frequency of the heart rate (1×HR). The signal is then passed through a slew-rate filter, at 235, to eliminate signals that have slew-rates that vary more than a predetermined percentage from one pulse to the next pulse. For example, a slew rate of about ±25% from one-pulse to the next may be selected for the slew-rate filter to reduce or eliminate signals from pulses that vary more than ±25% pulse-to-pulse.

At 237, the signal passes through a first high-pass filter. In an embodiment, the first high-pass filter has a p-value of 0.95. In one embodiment, all of the preceding operations are carried out at 1×HR. At operation 239, the signal is up-sampled. Up-sampling reduces artifacts (beat effects due to phase sensitivity) that would otherwise occur in the waveform as β approaches 0.5. An increased sampling frequency therefore captures proper phase information and therefore reduces or eliminates possible issues due to phase. In a specific exemplary embodiment, the signal is up-sampled to about double the frequency at 2×HR. The up-sampled frequency is then sent through a second high-pass filter at 241. In an embodiment, the second high-pass filter has a p-value of 0.50.

Front-end processed signals of pT₀ and pM₀ are output at 243, 263, respectively, from the digital-signal filtering methods 230, 250.

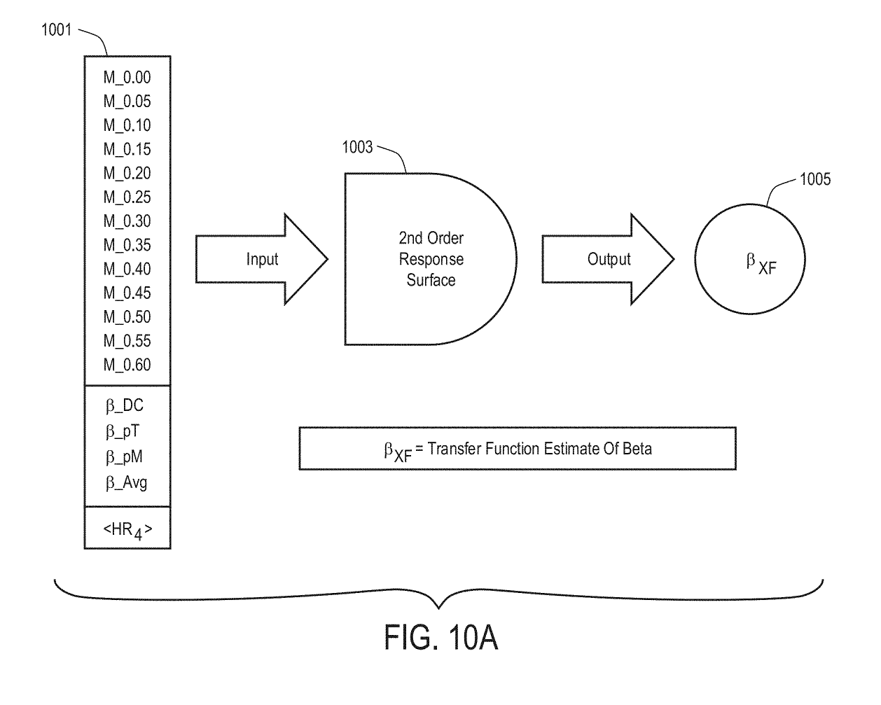

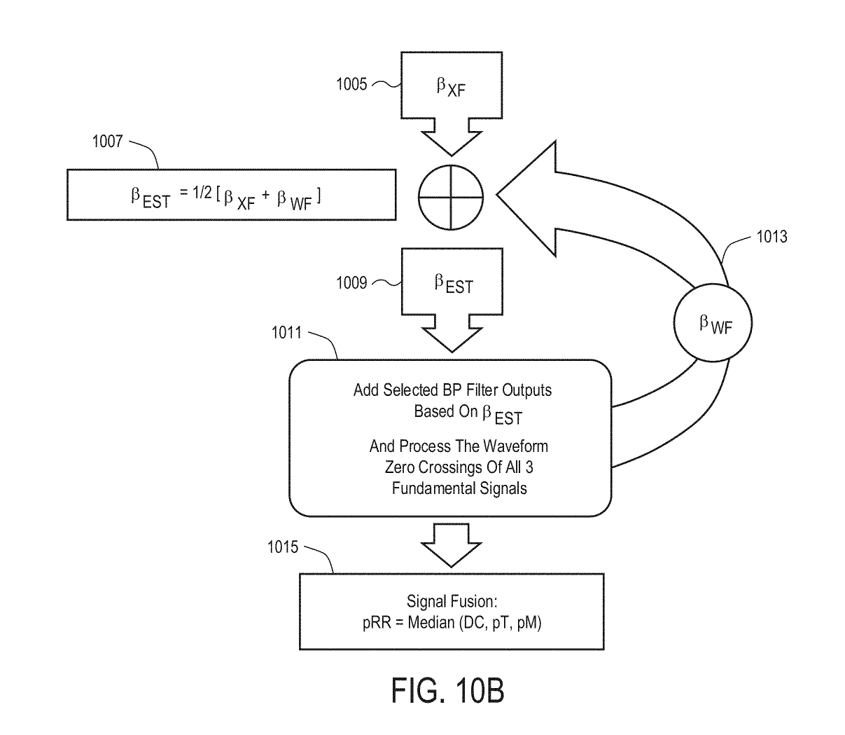

In addition to the three front-end processed signals of DC₀, pT₀, and pM₀, a fourth fundamental input used in later processing, discussed below with reference to FIG. 10A, is a four-beat average heart rate, <HR₄>. The four-beat average heart rate is extrapolated from the up-sampled 2(HR) frequency used at 239, 259. Upon reading and understanding the disclosure provided herein, the skilled artisan will recognize that the fourth fundamental input may be chosen to be other values as well. For example, the fourth fundamental input may be selected to be another integral value of the heart rate.

After this point, all further signal processing is performed in pulse time and not in real time. By performing all additional calculations in pulse time, fewer numbers of bandpass filters can be used since a total calculation range is determined quickly by relying on pulse-time calculations. The signal bandwidth of interest is then determined automatically by using the pulse-time calculations.

The skilled artisan will have recognized the use of the two high-pass filters in each of FIGS. 2A through 2C. Utilizing a two-pole high-pass filter helps significantly reduce or eliminate low-frequency noise caused by Mayer waves and other sources.

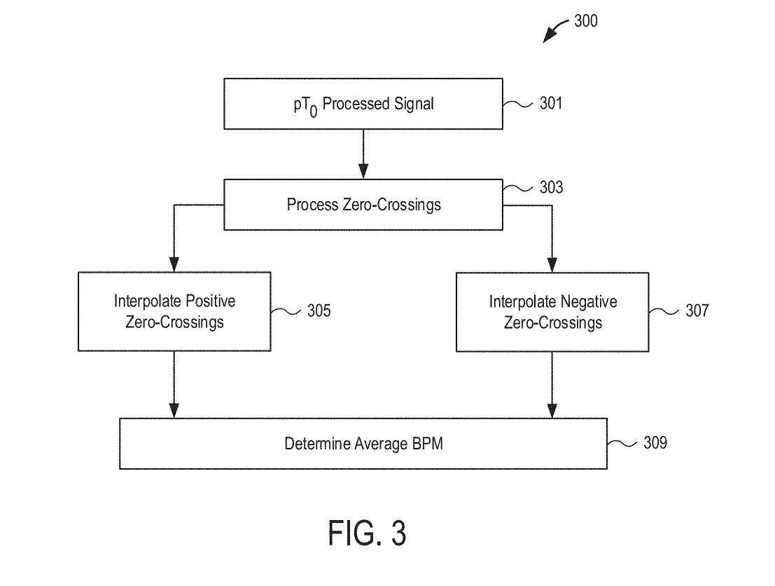

Referring now to FIG. 3, a fixed-filter algorithm 300 is shown. At 301, the pT₀ processed signal is processed for zero-crossings at 303. The skilled artisan will recognize, with reference again to FIG. 1D, that only the frequency-modulated pT signal will have any significant variation in frequency. Therefore, the fixed-filter algorithm 300 is applicable only to the pT signal, and consequently, to the front-end processed pT₀ signal as well since each has varying frequencies of zero-crossings.

With continued reference to FIG. 3, the pT₀ processed signal is processed, using, for example, an interpolated zero-crossing filter, for zero-crossings at 303 by considering both the positive (rising) edges and the negative (falling) edges of the signal. A distance between the interpolated zero-crossings indicates the period, and, consequently, based on the period, the breath rate.

At 305, 307, an interpolation of positive and negative zero-crossings is determined and a median value of the breath rate is calculated for each of the positive and the negative edges. Calculation of the median value can be considered an application of the median filter.

In an embodiment, the median period of the breath rate, based on the positive zero-crossings and determined from considering three breaths up to nine breaths, is given by equation (2):

<bT>_{P}=bT Median[3,9](sec) (2)

The median period of the breath rate, based on the negative zero-crossings, is given by equation (3):

<bT>_{P}=bT Median[3,9](sec) (3)

In this embodiment, a three-breath minimum is used to eliminate outliers in a subject's breathing pattern and, consequently, increase accuracy of the determined breath rate by reducing noise caused by breath-to-breath variations in the subject's breathing. The nine-breath maximum was determined experimentally as providing a consistent median value of breath rate that is consistent with a subject's actual breathing rate in most subjects. Additional experimental measurements have determined that some subjects have extremely consistent pulse rates—pulse rates have been observed within a root-mean-square (RMS) variation as small as three milliseconds and as large as 60 msec. However, the inventive subject matter described herein has been established based on being applicable to the entire population.

Equations (2) and (3) typically produce slightly different results since both the phase is slightly different and the duty cycle is changing.

The average breaths per minute, BPM_{P}, based on the breath time between the positive zero-crossings, is given by equation (4):

<BPM>_{P}=60/<bT>_{P} (4)

In a similar fashion, average breaths per minute, BPM_{N}, based on the breath time between the positive zero-crossings, is given by equation (5):

<BPM>_{N}=60/<bT>_{N} (5)

The average number of breaths per minute, BPM, is then determined as an arithmetic average of the positive average breaths per minute, BPM_{P} and BPM_{N}, according to equation (6):

<BPM>=½[<BPM>_{P}+<BPM>_{N}] (6)

By determining the approximate breath rate by processing and calculating zero-crossings as shown above, an initial time to first display the breath rate of the subject, in accordance with this embodiment, occurs after only four positive edges and four negative edges. The time to display the breath rate with most subjects then is approximately 15 seconds. Further, the computational requirements are very limited. For example, a processor with a limited computational speed can readily perform the calculations shown above to determine and display an initial approximation of breath rate of a subject. However, the approximation of breath rate is still accurate with little variation from much more involved methodologies, for example, as described with reference to the adaptive-filtering techniques, below.

As discussed above with regard to FIG. 2A, FIG. 4 shows a method 400 for determining how a running DC average is calculated over two pulse lengths. A first pulse 401 and a second pulse 403 are divided, in time, into a first-half pulse time 401A, 403A, respectively, as well as a second-half pulse time 401B, 403B, respectively. A total summation, S, of the number of samples, N, is shown for each half pulse, shifted by one-half pulse at a time. Thus, the first half of the first pulse 401 has a total number of samples, N_{1B}, of the total number of samples, N₁, for the entire first pulse, is ½ of the total number of samples and is calculated as:

N_{1A}=N₁/2

Similarly, the second half of the first pulse 401 has a total number of samples, N_{1B}, of the total number of samples, N₁, for the entire first pulse, is ½ of the total number of samples and is calculated as:

N_{1B}=N₁/2

Similar calculations are made for the second pulse 403, with each summation being shifted one-half pulse at a time. A skilled artisan will immediately recognize that other portions of the pulses can be determined and calculated that are not ½ pulse portions being unpatentable over, rather, some other fractional amount or amounts.

From this information, a running DC average, determined as a continuous function <F>, for each time, t, in a period, T, over a predefined number of pulses is then calculated as:

$$〈 F 〉 = \frac{{\int }_{0}^{T} F ( t ) dt}{T}$$

For the discrete values sampled, an average DC signal for each of the Y pulse incremental ranges, 405, 407, 409, shown in FIG. 4 at twice the pulse rate can be calculated for each ½ pulse increment as:

$${〈 D C 〉}_{0} = \frac{( {S}_{1 A} + {S}_{1 B} )}{( {N}_{1 A} + {N}_{1 B} )}$$

$${〈 D C 〉}_{1} = \frac{( {S}_{1 B} + {S}_{2 A} )}{( {N}_{1 B} + {N}_{2 A} )}$$

$${〈 D C 〉}_{2} = \frac{( {S}_{2 A} + {S}_{2 B} )}{( {N}_{2 A} + {N}_{2 B} )}$$

Thus, <DC>₀ is calculated as the average of the half-pulse summations divided by the number of samples over the entire first pulse, <DC>₁ is calculated as the average of the half-pulse summations over the second half of the first pulse and the first half of the second pulse, divided by the number of samples over that pulse range, and <DC>₂ is calculated as the average of the half-pulse summations by the number of samples over the entire second pulse.

Consequently, at twice the heart rate, the average signal of DC₀ 411 for the PPG can be determined. When combined with the high-pass filtering shown and described above with reference to FIG. 2A, a resultant transfer function does not depend on a real-time frequency. The resultant transfer function depends only on β. Thus, any influence of a real-time heart rate is eliminated and all calculations are determined purely in pulse time.

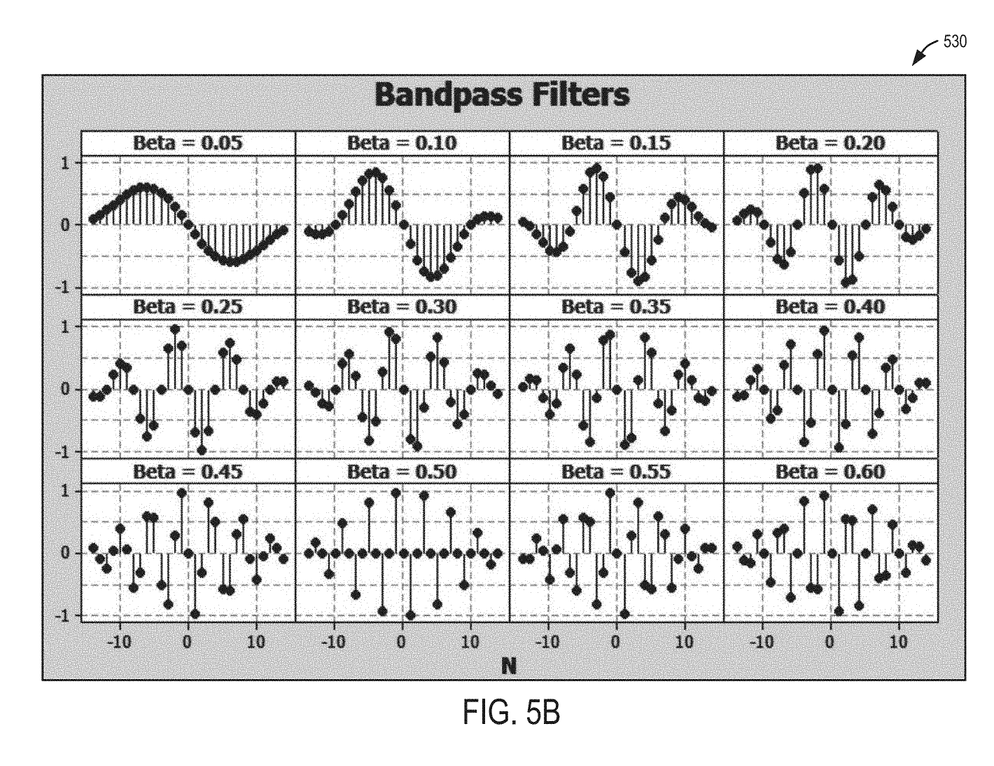

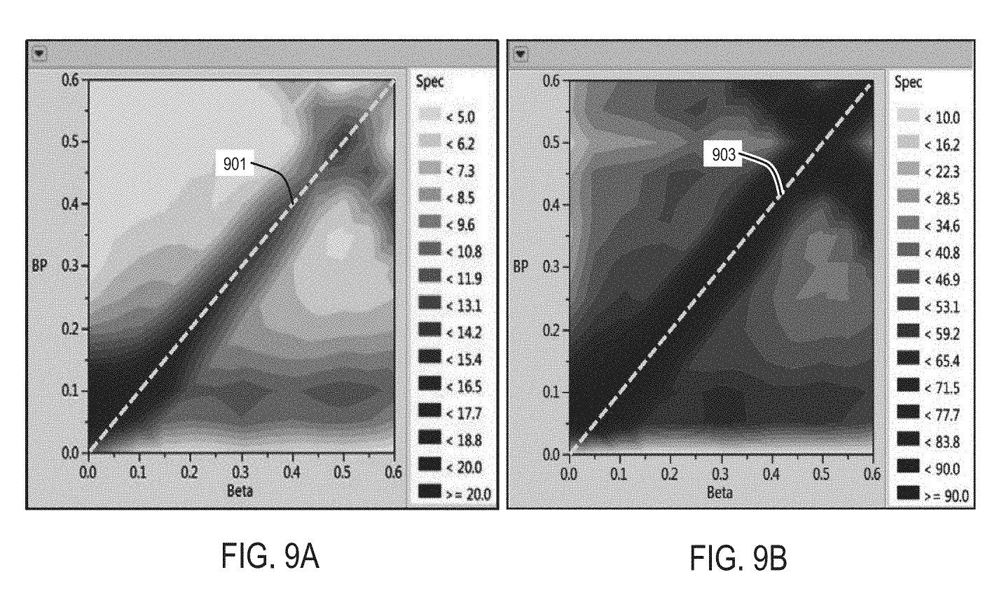

With reference now to FIGS. 5A and 5B, adaptive-filtering elements of the inventive subject matter are shown. In an embodiment, thirteen bandpass filters are employed—one bandpass filter for each value of β from 0.00 to 0.60, incremented by a step size of 0.05. A β value of 0.60 is the maximum chosen since aliasing will otherwise occur much above β=0.5. As noted above, any frequency content above the Nyquist frequency may encounter aliasing errors. The aliasing error is shown and discussed with reference to FIGS. 9A and 9B, below.

The graph 530 of FIG. 5B shows a normalized intensity plot for outputs from each of the 13 linear-phase bandpass filters, starting at β=0, for each of 29 taps (e.g., samples at ±14 plus a zero-point). Each of the bandpass filters is running constantly for the adaptive filter and provide a rough estimate of an actual value of β for a given signal. By selecting an appropriate value of β, from one or more of the bandpass filters, a correct adaptive filter can be selected as described in more detail below. Consequently, the bandpass filter helps determine the proper value of β in a frequency domain. Once the maximum signal strength is found for a given value of β for a single bandpass filter, the spectral output from the selected bandpass filter is added to the output of the two nearest-neighbor bandpass filters (at β_{-0.05} and β_{+0.05}) in order to open up the bandwidth to later determine the real zero-crossing from the PPG waveform. As understood by the person of ordinary skill in the art, the addition of the spectral outputs is possible works because the filters are “linear phase.” Consequently, all frequencies see the same phase lag across each of the 13 band-pass filters.

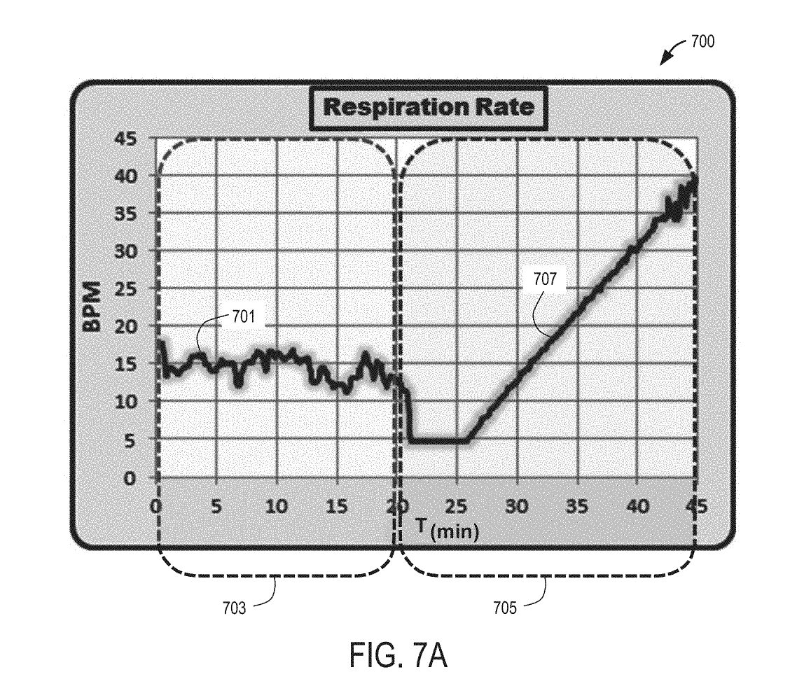

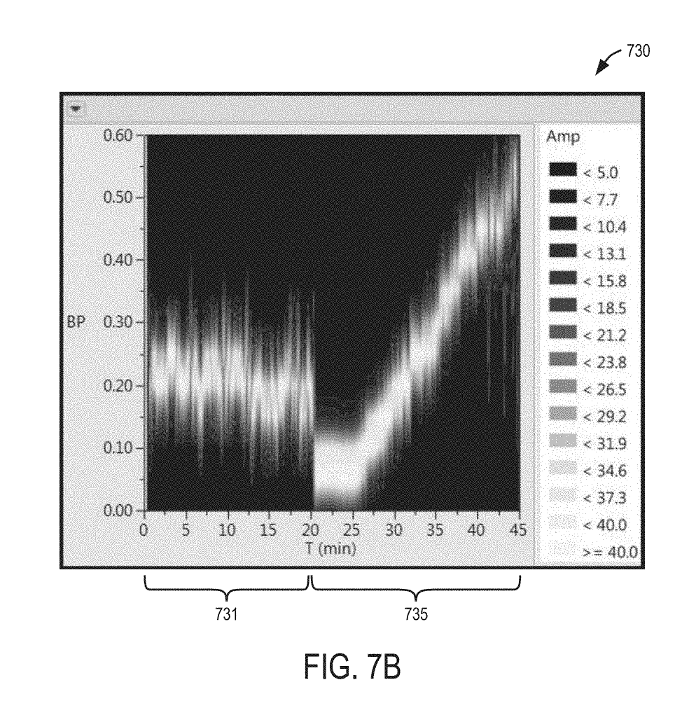

A skilled artisan will recognize that a smaller number or a larger number of bandpass filter may be employed for finding the central frequency of a signal. A smaller number of bandpass filters increases computational speeds with some sacrifice in accuracy. A smaller number of bandpass filters will also have an effect on FIG. 5A since the overlap from each bandpass filter with adjacent bandpass filters will be lessened. However, based on actual clinical testing, a larger number of bandpass filters will increase computational time but will not necessarily result in a concomitant increase in accuracy. Results of the clinical comparisons of calculated values of respiration rate derived from the PPG are shown and discussed with regard to FIGS. 7A through 9B, below.

Referring again to FIG. 5, the graph 500 shows a plot of the intensity of the signal value, H(β), as a function of β for each of the 13 bandpass filters. The value of H shows different values of the center frequency β for β=0.00 to 0.60 in increments of 0.05. H is determined as a function of both β and the number of taps in the bandpass filter, n. (Recall that β is defined as the breath frequency when sampling at the pulse rate.) H[n, β] can be determined from the following equation:

$$H [ n , β ] = sin ( πβ n ) * {cos}^{2} ( \frac{π n}{36} ) \qquad ( 7 )$$

A skilled artisan will recognize that a cosine-squared windowing function is employed by equation (7) to reduce or eliminate any or most spectral leakage.

In this embodiment, each bandpass filter is a Type 2 (odd/anti-symmetric) linear phase filter. As noted above, each bandpass filter has 29 taps at twice the heart rate—extending over 14.5 pulses and having the same phase delay at all frequencies. Since the bandpass filters, by virtue of being linear phase filters, or approximately a linear-phase filter, each have the same phase delay, outputs from each of the bandpass filters can be added directly. Being able to add outputs directly can save considerable computational time as will be discussed in more detail below.

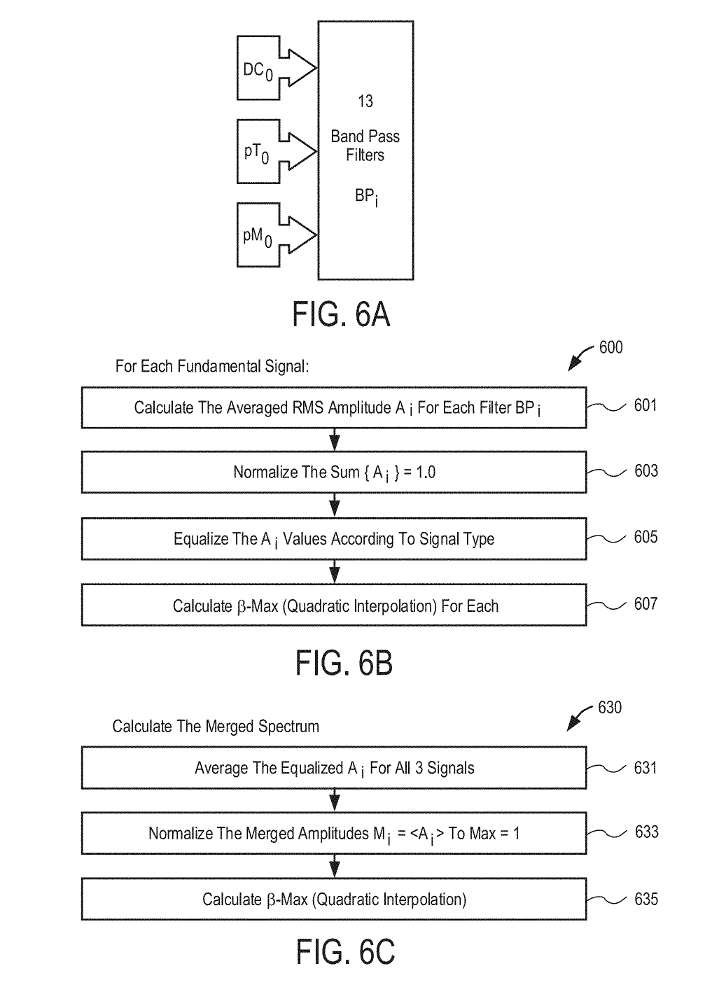

FIGS. 6A through 6C show additional operations for determining a respiration rate using the adaptive-filter algorithm. As shown in FIG. 6A, each of the three fundamental signals, DC₀, pT₀, and pM₀, are input separately into each of the 13 bandpass filters, BPᵢ, where I=13 in this embodiment. (Recall that the three fundamental signals, DC₀, pT₀, and pM₀ are described above with reference to FIGS. 2A through 2C.)

Referring now to the method 600 of FIG. B, for each of the three fundamental signals, at 601, the averaged RMS amplitude, Aᵢ, output from each filter, BPᵢ, is calculated. The summation for each calculated value of Aᵢ is then normalized to 1.0 for further processing at 603. At 605, each of the Aᵢ values is equalized according to the three fundamental signal types. A quadratic interpolation is then performed to calculate β_{MAX} for each of three fundamental signal types. Consequently, the method 600 determines at what value of β is the peak response present for each of the three fundamental signal types. The determined and calculated value for β_{MAX} for each of three fundamental signal types is then combined into a merged spectrum for further processing.

The method 630 of FIG. 6C calculates a β_{MAX} value for the merged spectrum. At 631, the equalized value of Aᵢ is equalized for each of three fundamental signal types. The merged amplitude values are normalized to a maximum value of “1” at 633. A calculated value of β_{MAX,AVG} is calculated utilizing a quadratic interpolation from the merged spectrum at 635.

From the methods 600, 630 of FIGS. 6B and 6C, four new inputs are produced for further processing as shown and described with reference to FIGS. 10A and 10B, below. The four new inputs produced are the determinations of β_{MAX} based on the three fundamental signal types, β_{DC}, β_{pT}, and β_{pM}. The fourth input β_{MAX,AVG}, is the spectral estimate of β based on the maximum RMS amplitude measured by the band-pass filters for the averaged spectrum along with the value of β_{MAX} from the three fundamental signal types, β_{DC}, β_{pT}, and β_{pM}.

As an example of applying the method, there is an equalized spectrum for each of the three fundamental signals (pT, pM, and DC), and also a merged spectrum, each of which is comprised of the 13 band-pass filters. For each of those four spectra, a β_{MAX} value is calculated every half pulse as follows:
Find the band-pass filter that has the largest RMS amplitude. For example, if the band-pass filter at β=0.20 has a maximum RMS amplitude, then the value of β is close to 0.2, which would be the first-order estimate that is quantized in steps of 0.05.
Using the maximum RMS amplitude and also the RMS amplitudes of the two nearest band-pass filters (in this example, the band-pass filter and the two-nearest neighbors would be 0.15, 0.20, and 0.25), a quadratic interpolation of the RMS amplitudes is performed to estimate the actual location of the peak RMS amplitude from which a refined estimate of β is then calculated (e.g., β_{MAX}=0.22 in this example, which would be the second-order estimate).

For verification of the inventive subject matter, a determination was made whether the calculated values of β, both from the fixed-filtering algorithm of FIG. 3, as well as the initial steps of the adaptive-filtering algorithm of FIGS. 6A through 6C, accurately represent an actual respiration rate of a subject under test. Referring to FIG. 7A, a respiration rate graph 700 showing Breaths per Minute (BPM) as a function of time is shown. During an initial 20-minute period 703, the line 701 indicates the normal respiration rate, in BPM, of a subject. However, during the last time period 705, from 20 minutes to 45 minutes, the subject was asked to breathe in unison with a metronome that was varied to produce from 5 to 40 clicks per minute. The subject's respiration rate during the last time period 705 is shown by line 707.

A time-dependent spectrum graph 730 of FIG. 7B shows an example of true values of 3 obtained from a capnograph measurement of carbon dioxide (CO₂) values during exhalations from the subject. The β values of the time-dependent spectrum graph 730 show each of the 13 discrete bandpass filter amplitudes as a function of time and were determined by a quadratic interpolation of the bandpass filter amplitudes. The ordinate axis of the graph 730 indicates an output from each of the 13 bandpass filters (0.00 to 0.60 with intermediate tick marks at 0.05). The gray-scale amplitude of each bandpass filter is indicated by the “Amp” scale on the right-side of the graph 730. The lighter-values of amplitude indicate a center peak value of β for the breath rate versus time. Although the graph seems to indicate a continuous plot, there is actually only a single vertical line for each bandpass filter per time interval. Consequently, the graphs 700, 730 can be used to train the algorithms described herein. Thus, high-pass filtering reduces Mayer waves, however, if the p-values are set too low, the signal is also reduced. Consequently, the p-values are chosen to maximize SNR.

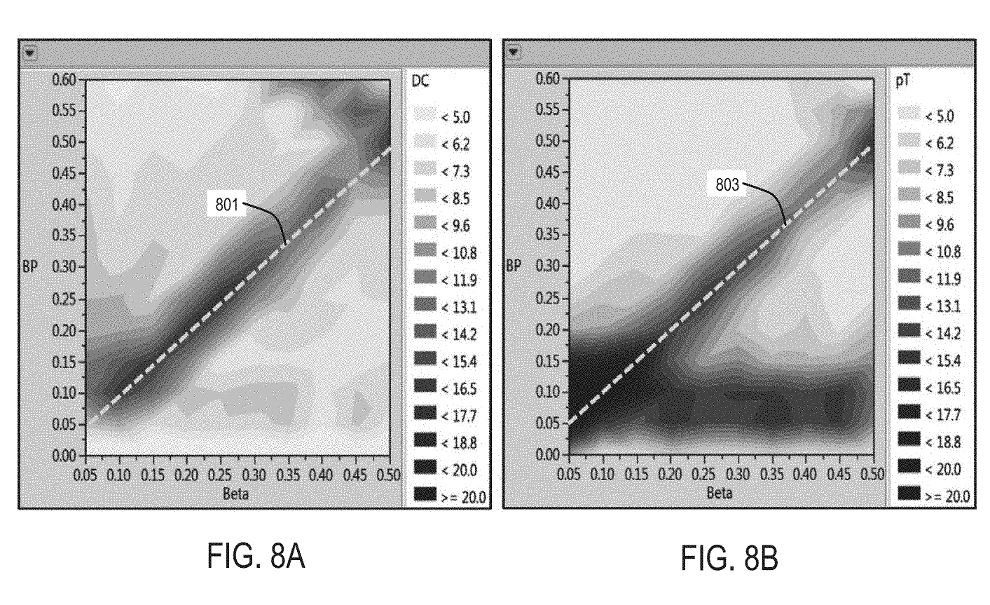

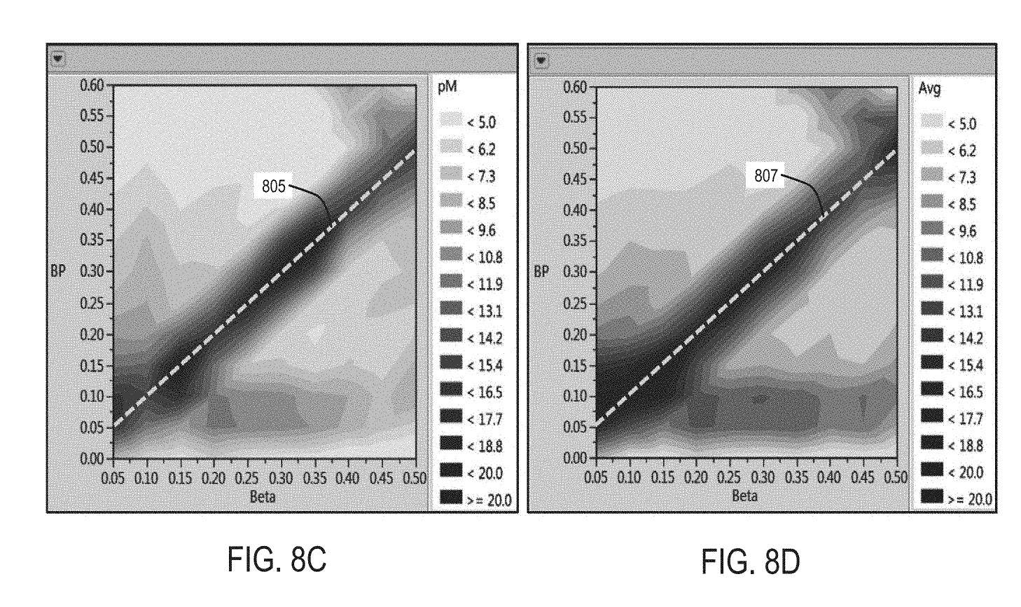

FIGS. 8A through 8D show separate examples of the spectra for each of the fundamental signals for one subject, plus the average of the three signals as discussed with reference to FIGS. 6B and 6C, plotted against a true value of β. For example, FIG. 8A shows an intensity graph of the DC fundamental signal for each of the 13 bandpass filter values, 0.00 to 0.60, on the ordinate axis compared by a regression line 801 against the true value of β as indicated on the abscissa. FIG. 8B shows an intensity graph of the pT fundamental signal for each of the 13 bandpass filter values on the ordinate axis compared by a regression line 803 against the true value of β on the abscissa. FIG. 8C shows an intensity graph of the pM fundamental signal for each of the 13 bandpass filter values on the ordinate axis compared by a regression line 805 against the true value of β on the abscissa. Finally, FIG. 8D shows an intensity graph of the average spectrum of each of the three fundamental signal spectra for each of the 13 bandpass filter values on the ordinate axis compared by a regression line 807 against the true value of β on the abscissa.

The skilled artisan will detect some deviation from the regression lines 801, 803, 805, 807 at approximately 0.05 to 0.10 on the ordinate axis. These deviations are caused by Mayer waves, discussed above with reference to FIG. 1B. An intensity level of the Mayer waves can vary tremendously from subject-to-subject. Consequently, reducing or eliminating any effects from Mayer waves is at least one of the reasons for an application of a two-pole high-pass filter as described above with reference to FIGS. 2A through 2C. Any intensity value variation in Mayer waves from one subject to another (e.g., subject bias) will be reduced or eliminated.

FIGS. 9A and 9B show the impact of spectral equalization prior to averaging three fundamental spectra as shown with reference to FIGS. 8A through 8C. FIG. 9A shows a regression line 901 comparing the bandpass filter output to true β for a subject average-spectrum. FIG. 9B shows a regression line 903 comparing the bandpass filter output to true β for a subject average-equalized-spectrum. Thus, with reference to the intensity scale on the right side of FIGS. 9A and 9B, the amplitude at β=0.4 is a much higher intensity value in the subject average-equalized-spectrum of FIG. 9B. Thus, equalizing the average spectrum increases still further the accuracy of a calculated β versus the true β.

The skilled artisan will also note the “t-shaped” spread in the spectra of a β value of approximately 0.5. The spread is due to an aliasing effect as described herein. However, as also described herein with regard to most human subjects, a typical respiration rate is much less than one-half the heart pulse rate. Therefore, the aliasing effect seldom, if ever, has an impact in calculating a value of β for a given subject.

FIG. 10A provides additional details on increasing the accuracy of β as determined by the adaptive-filter algorithm based on using a number of inputs 1001 to develop a second-order surface response function 1003. An output of the second-order surface response function 1003 is then used to determine a transfer function estimate of β, β_{XF}, at 1005. In this embodiment, all inputs and outputs are functions of time sampled in pulse time at a frequency, F_{S}, equal to two times the heart rate.

The inputs 1001 include the normalized Merged spectral amplitudes or “M” values (M_{0.00} to M_{0.06}) of the outputs of the 13 bandpass filters from the merged spectrum as shown and described with reference to FIG. 6C; the four different estimates of β (β_{DC}, β_{pT}, β_{pM}, and β_{MAX, AVG}); and the four-beat average heart rate, <HR₄>.

The inputs 1001 are input to the second-order surface response function 1003. In an embodiment, 45 terms (based on the 18 input-factors as noted immediately above) are used to calculate an output of the second-order surface response function 1003, an output of which is the transfer function estimate of β, β_{XF}, 1005. The determined transfer function estimate β_{XF} indicates the center value of the signal, β, for choosing the adaptive filer.

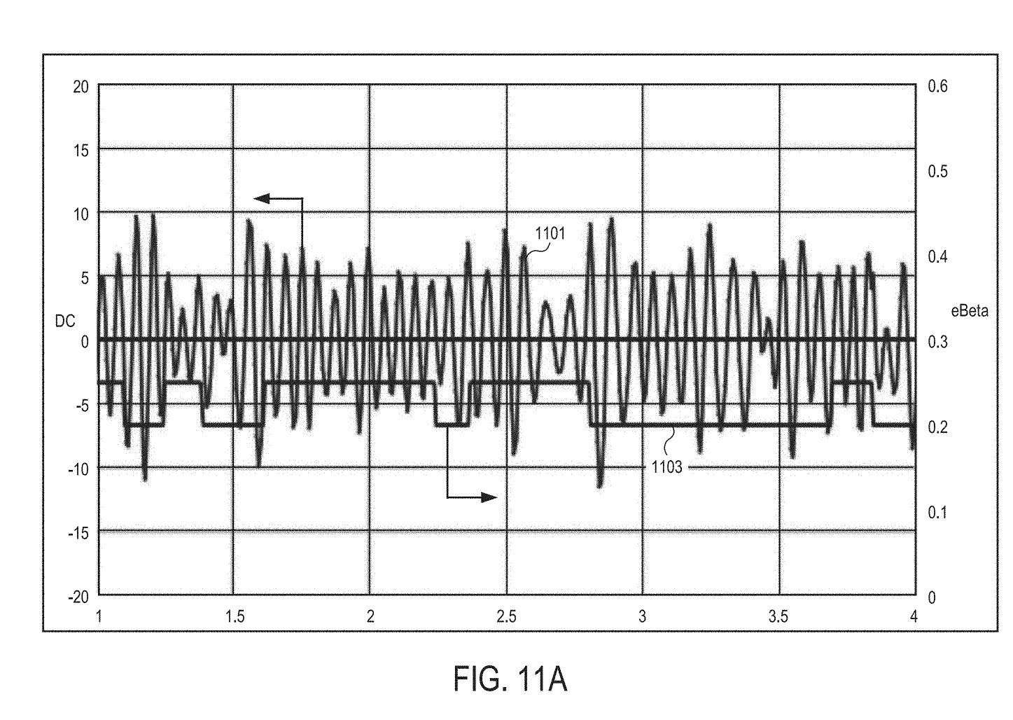

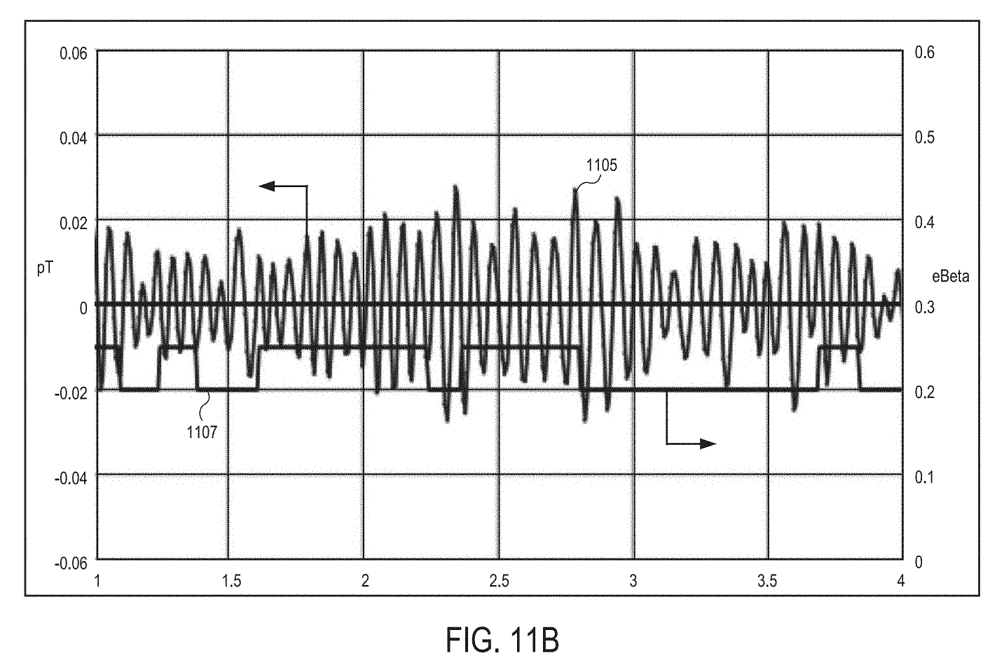

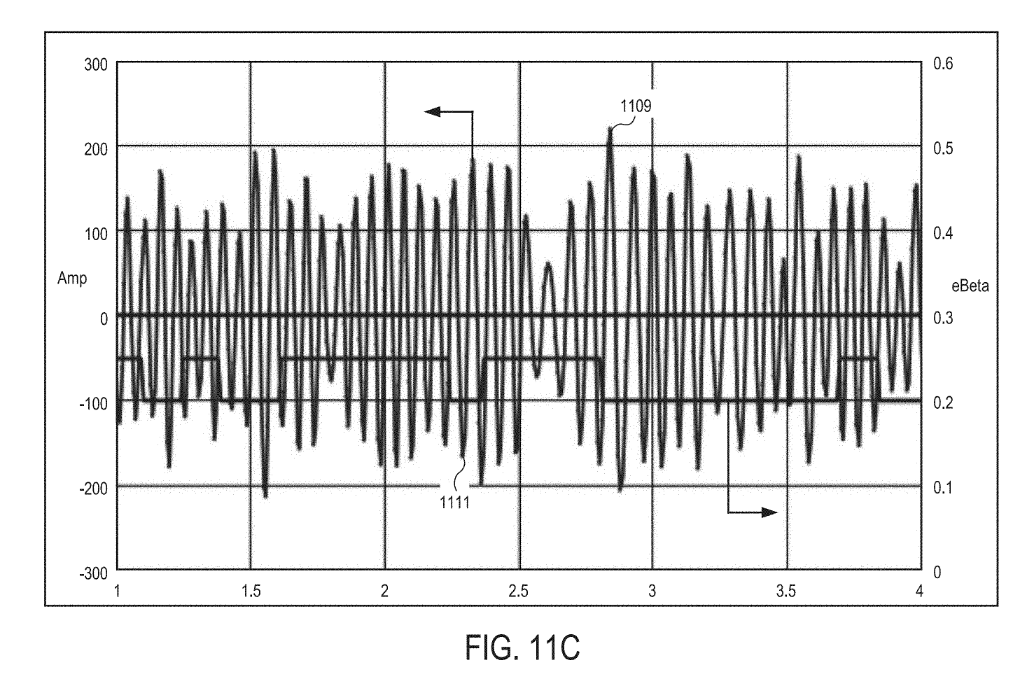

Referring again to the second-order surface response function 1003, the skilled artisan will recognize that, based on the 18 input values, 190 factors can be calculated. For example, considering only a two-factor input, i₁ and i₂, the surface response function would include a first-order function, i₁+i₂. The second order response function would include i₁+i₂, i₁×i₂, i₁², and i₂². As such, a response surface methodology (RSM), in general, considers relationships between a number of input variables and one or more resulting response variables. The RSM can be used in a design-of-experiments to estimate an optimal response function. The skilled artisan will further recognize that a larger or smaller number of factors may be employed depending upon a desired accuracy of β. Waveform examples with dynamic β estimates are described and shown with reference to FIGS. 11A through 11C, below.

In FIG. 10B, the spectral and time-domain estimates of β are combined to produce a nonlinear enhancement of resolution of an actual value of β. With concurrent reference to FIGS. 11A through 11C, the transfer function estimate of β, β_{XF}, 1005, is initially used to determine an estimated value of β, β_{EST}, at 1009. At 1011, the current value of β_{EST} is combined with selected bandpass filter outputs, as described and shown in FIGS. 11A through 11C, below. The waveform zero-crossings from all three fundamental signals, DC, pT, and pM, are used to determine an additional β value based on the waveforms, β_{WF}. The new estimate based on the waveforms, β_{WF}, is then fed back at 1013. A new β_{EST} value is calculated at 1007 as the arithmetic average of the original transfer function estimate of β, β_{XF}, and the waveform estimated value of β, β_{WF}. Therefore, the new estimate of β is determined as:

β_{EST}=½[β_{XF}+β_{WF}]

In a specific exemplary embodiment, once the new estimate based on the waveforms, β_{WF}, is then fed back at 1013 (e.g., about 15 pulses after the first estimate of β_{XF}), the “loop” runs continuously in time. At 1015, a signal fusion occurs where the predicted respiration rate, pRR, is determined as a median value of the respiration rates as determined for each of the three signals DC, pT, and pM, as described and shown with reference to FIGS. 11A through 11C.

FIGS. 11A through 11C show waveform examples with dynamic 13 estimates (ebeta) in pulse time. FIG. 11A shows a plot of the DC waveform 1101 with an estimate of β 1103 changing based on the DC waveform. FIG. 11B shows a plot of the pT waveform 1105 with an estimate of β 1107 changing based on the pT waveform. And FIG. 11C shows a plot of the pM waveform 1109 with an estimate of β 1111 changing based on the DC waveform.

In an embodiment, when an estimate of β has been determined in accordance with various aspects of the inventive subject matter described herein, the bandpass filter closest to the estimate, along with two-nearest neighbors (that is, a total of three bandpass filters), are utilized in processing the waveform for a given signal type. The zero-crossings (considering both positive-edge zero-crossing and negative-edge zero-crossings) may then be used to determine the respiration rates of a subject. Along with consideration of the three fundamental signals, the actual zero-crossings can provide yet a further estimate of the actual value of β. The combined-β estimate (transfer function plus feedback) performs better than either value used alone. As described with reference to FIG. 10B, above, this estimate of β determined from the zero-crossings may be fed back at 1013 (FIG. 10B) to provide an enhanced (more accurate) estimate of β. Therefore, the active feedback loop 1013 provides a continually-updating estimate of the actual value of β. A fusion of the three signals, based on the continually-updating estimate of the actual value of β of the three waveforms, allows a choice of the best adaptive filter by maximizing the overall signal-to-noise ratio, thereby providing a higher-level of accuracy.

Clinical trials have indicated that accurate values of β, and consequently respiration rate, can be determined quickly and accurately. For example, using the fixed (non-adaptive) filter algorithm described and shown with reference to FIG. 3, an accurate determination of respiration rate, in breaths per minute (BPM) can be determined accurately in approximately 15 seconds. When needed for a particular subject (e.g., due to a clinical requirement for a highly-accurate value of BPM or in a case where a subject may have several “noise” contributing factors as described herein), an even more accurate estimate of BPM can be determined by the adaptive-filtering methods described herein. For a highly-accurate level of BPM, the adaptive-filtering methods described herein can be determined in approximately 45 seconds. Additionally, the skilled artisan will recognize that not all steps of the adaptive-filtering algorithm need to be utilized depending on an accuracy level required for a given subject.

### Exemplary Machine Architecture and Machine-Readable Storage Medium

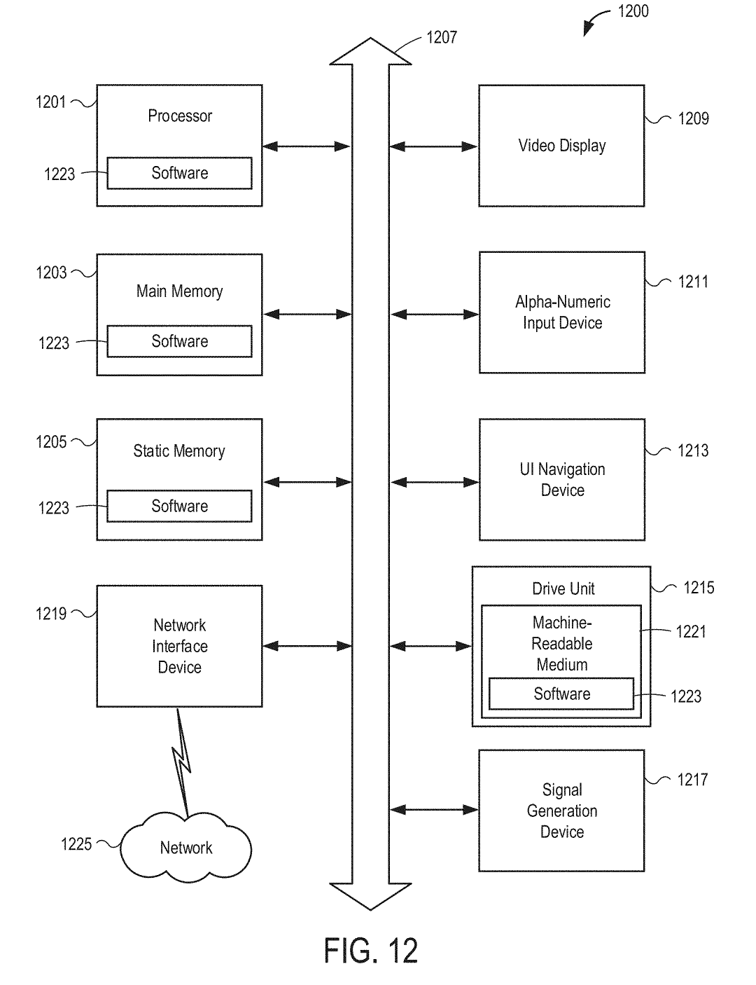

With reference now to FIG. 12, an exemplary embodiment extends to a machine in an example of a computer system 1200 within which instructions, for causing the machine to perform any one or more of the methodologies discussed herein, may be executed. In alternative exemplary embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, a switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1200 includes a processor 1201 (e.g., a hardware-based microprocessor or embedded hardware-based processor, a hardware-based central processing unit (CPU), a hardware-based graphics processing unit (GPU), or various combinations thereof), a main memory 1203 and a static memory 1205, which communicate with each other via a bus 1207. The computer system 1200 may further include a video display unit 1209 (e.g., a liquid crystal display (LCD) or a cathode ray tube (CRT)). The computer system 1200 also includes an alphanumeric input device 1211 (e.g., a keyboard), a user interface (UI) navigation device 1213 (e.g., a mouse), a disk drive unit 1215, a signal generation device 1217 (e.g., a speaker), and a network interface device 1219.

### Machine-Readable Medium

The disk drive unit 1215 includes a non-transitory machine-readable medium 1221 on which is stored one or more sets of instructions and data structures (e.g., software 1223) embodying or used by any one or more of the methodologies or functions described herein. The software 1223 may also reside, completely or at least partially, within the main memory 1203 or within the processor 1201 during execution thereof by the computer system 1200; the main memory 1203 and the processor 1201 also constituting machine-readable media.

While the non-transitory machine-readable medium 1221 is shown in an exemplary embodiment to be a single medium, the term “machine-readable medium” may include a single medium or multiple media (e.g., a centralized or distributed database, or associated caches and servers) that store the one or more instructions. The term “non-transitory machine-readable medium” shall also be taken to include any tangible medium that is capable of storing, encoding, or carrying instructions for execution by the machine and that cause the machine to perform any one or more of the methodologies of the present invention, or that is capable of storing, encoding, or carrying data structures used by or associated with such instructions. The term “non-transitory machine-readable medium” shall accordingly be taken to include, but not be limited to, solid-state memories, and optical and magnetic media. Specific examples of machine-readable media include non-volatile memory, including by way of exemplary semiconductor memory devices (e.g., EPROM, EEPROM, and flash memory devices); magnetic disks such as internal hard disks and removable disks; magneto-optical disks; and CD-ROM and DVD-ROM disks.

### Transmission Medium

The software 1223 may further be transmitted or received over a communications network 1225 using a transmission medium via the network interface device 1219 utilizing any one of a number of well-known transfer protocols (e.g., HTTP). Examples of communication networks include a local area network (LAN), a wide area network (WAN), the Internet, mobile telephone networks, Plain Old Telephone (POTS) networks, and wireless data networks (e.g., WiFi and WiMax networks). The term “transmission medium” shall be taken to include any intangible medium that is capable of storing, encoding, or carrying instructions for execution by the machine, and includes digital or analog communications signals or other intangible medium to facilitate communication of such software.

Included in the disclosed subject matter provided herein are various system and method diagrams describing various embodiments of the particulate matter sensor calibration system. Therefore, the description above includes illustrative examples, devices, systems, and methods that embody the disclosed subject matter. In the description, for purposes of explanation, numerous specific details were set forth in order to provide an understanding of various embodiments of the inventive subject matter. It will be evident, however, to those of ordinary skill in the art that various embodiments of the inventive subject matter may be practiced without these specific details. Further, well-known structures, materials, and techniques have not been shown in detail, so as not to obscure the various illustrated embodiments. For example, the skilled artisan will recognize that each of the filtering algorithms described herein can be implemented in hardware, software, firmware, or various combinations thereof. Also, the various filters can be analog filters in addition to digital filters, or a combination of the two.

In accordance with the present disclosure, components, process steps, and/or data structures may be implemented using various types of operating systems, programming languages, computing platforms, computer programs, and/or general-purpose machines. In addition, those of ordinary skill in the art will recognize that devices of a less general purpose or nature, such as hardwired devices, field programmable gate arrays (FPGAs), application specific integrated circuits (ASICs), or the like, may also be used without departing from the scope of the concepts disclosed herein. For example, the skilled artisan will recognize that one or more of the filter described herein can be implemented in an FPGA device. As also described herein, various embodiments may be tangibly embodied as a set of computer instructions stored on a computer readable medium, such as a memory device.

As used herein, the term “or” may be construed in an inclusive or exclusive sense. Additionally, although various exemplary embodiments discussed herein focus on particular ways to determine an estimate of β, other embodiments will be understood by a person of ordinary skill in the art upon reading and understanding the disclosure provided. Further, upon reading and understanding the disclosure provided herein, the person of ordinary skill in the art will readily understand that various combinations of the techniques and examples provided herein may all be applied in various combinations.

Although various embodiments are discussed separately, these separate embodiments are not intended to be considered as independent techniques or designs. As indicated above, each of the various portions may be inter-related and each may be used separately or in combination with other particulate matter sensor calibration system embodiments discussed herein.

Consequently, many modifications and variations can be made, as will be apparent to the person of ordinary skill in the art upon reading and understanding the disclosure provided herein. Functionally equivalent methods and devices within the scope of the disclosure, in addition to those enumerated herein, will be apparent to the skilled artisan from the foregoing descriptions. Portions and features of some embodiments may be included in, or substituted for, those of others. Such modifications and variations are intended to fall within a scope of the appended claims. Therefore, the present disclosure is to be limited only by the terms of the appended claims, along with the full scope of equivalents to which such claims are entitled. It is also to be understood that the terminology used herein is for the purpose of describing particular embodiments only and is not intended to be limiting.

The Abstract of the Disclosure is provided to allow the reader to quickly ascertain the nature of the technical disclosure. The abstract is submitted with the understanding that it will not be used to interpret or limit the claims. In addition, in the foregoing Detailed Description, it may be seen that various features may be grouped together in a single embodiment for the purpose of streamlining the disclosure. This method of disclosure is not to be interpreted as limiting the claims. Thus, the following claims are hereby incorporated into the Detailed Description, with each claim standing on its own as a separate embodiment.

What is claimed is:

1. A system to determine a respiration rate of a subject from an output of a device capable of generating a photoplethysmogram (PPG) signal, the system comprising:
one or more hardware-based processors to sample the PPG signal at a first selected-frequency;
a first high-pass filter to filter the sampled PPG signal;
a second high-pass filter coupled in series with the first high-pass filter to receive an output from the first high-pass filter and to filter the output at a second selected-frequency;
a zero-crossing filter to receive an output from the second-high-pass filter and to interpolate positive-(rising) edge zero crossings and negative-(falling) edge zero crossings of at least a selected portion of the PPG signal to determine breath-time intervals caused by an influence of the respiration rate on the PPG signal; and
a median filter to determine an average of the breath-time intervals for the positive-edge zero-crossings and the negative-edge zero-crossings to derive an estimate of the respiration rate.

2. The system of claim 1, wherein the one or more hardware-based processors are further configured to separate components of the PPG signal into a DC-modulated waveform (DC signal), an amplitude-modulated waveform (pM signal), and a frequency-modulated waveform (pT signal).

3. The system of claim 2, wherein the first selected-frequency is a pulse-time frequency for the pM signal and the pT signal.

4. The system of claim 3, wherein the second selected-frequency is interpolated to about double the pulse-time frequency for the pM signal and the pT signal.

5. The system of claim 2, wherein the first selected-frequency is a real-time frequency for the DC signal.

6. The system of claim 2, wherein the second selected-frequency for the DC signal is the output of the first high-pass filter averaged over one pulse at a time and shifted by about one-half pulse at a time over a sampling pulse-time frequency of about two heart rate pulses.

7. The system of claim 1, wherein the one or more hardware-based processors are further configured to determine a pulse rate of the subject from the PPG signal.

8. The system of claim 1, further comprising a slew-rate filter to reduce effects or eliminate signals that have slew-rates that vary more than a predetermined percentage from one pulse to the next pulse.

9. The system of claim 8, wherein the predetermined percentage is about ±25%.

10. The system of claim 1, wherein the one or more hardware-based processors are to sample the PPG signal at a selected frequency based on a heart rate of the subject.

11. The system of claim 1, wherein at least one of the first high-pass filter and the second high-pass filter comprises a digital high-pass filter.

12. The system of claim 1, wherein at least one of the first high-pass filter and the second high-pass filter comprises an analog digital high-pass filter.

13. The system of claim 1, wherein the median filter is further configured to derive the estimate of the respiration rate based on calculating respiration rates for both the positive-edge zero crossings and the negative-edge zero crossings individually prior to averaging.

14. The system of claim 1, wherein the breath-time intervals are determined as a difference between interpolated zero-crossings.

15. A method for determining respiration rate of a subject from an output of a device capable of generating a photoplethysmogram (PPG) signal, the method comprising:
sampling the PPG signal at a first selected frequency;
filtering the PPG signal with a first high-pass filter;
receiving an output from the first high-pass filter and filtering the output at a second selected-frequency in a second high-pass filter;
receiving an output from the second-high-pass filter of at least a selected portion of the PPG signal;
interpolating positive-(rising) edge zero crossings and negative-(falling) edge zero crossings of the selected portion of the PPG signal to determine breath-time intervals caused by an influence of the respiration rate on the PPG signal; and
determining an average of the breath-time intervals for the positive-edge zero-crossings and the negative-edge zero-crossings to derive an estimate of the respiration rate.

16. The method of claim 15, further comprising separating components of the PPG signal into a DC-modulated waveform (DC signal), an amplitude-modulated waveform (pM signal), and a frequency-modulated waveform (pT signal).

17. The method of claim 16, wherein the first selected-frequency is a pulse-time frequency for the pM signal and the pT signal.

18. The method of claim 17, wherein the second selected-frequency is interpolated to about double the pulse-time frequency for the pM signal and the pT signal.

19. The method of claim 16, wherein the first selected-frequency is a real-time frequency for the DC signal.

20. The method of claim 16, wherein the second selected-frequency for the DC signal is the output of the first high-pass filter averaged over one pulse at a time and shifted by about one-half pulse at a time over a sampling pulse-time frequency of about two heart rate pulses.

21. The method of claim 15, further comprising determining a pulse rate of the subject from the PPG signal.

22. The method of claim 15, further comprising filtering the PPG signal with a slew-rate filter to reduce effects or eliminate signals that have slew-rates that vary more than a predetermined percentage from one pulse to the next pulse.

23. The method of claim 22, wherein the predetermined percentage is about ±25%.

24. The method of claim 15, wherein the sampling of the PPG signal is based on a selected frequency based on a heart rate of the subject.

25. The method of claim 15, wherein deriving the estimate of the respiration rate is based on calculating respiration rates for both the positive-edge zero crossings and the negative-edge zero crossings individually prior to averaging.

26. The method of claim 15, further comprising determining the breath-time intervals as a difference between interpolated zero-crossings.

27. A tangible computer-readable medium having no transitory signals and containing instructions that, when executed by one or more hardware-based processors of a machine, cause the machine to perform operations comprising:
determining a respiration rate of a subject from an output of a device capable of generating a photoplethysmogram (PPG) signal, the determination including
sampling the PPG signal at a first selected frequency;
filtering the PPG signal with a first high-pass filter;
receiving an output from the first high-pass filter and filtering the output at a second selected-frequency in a second high-pass filter;
receiving an output from the second-high-pass filter of at least a selected portion of the PPG signal;
interpolating positive-(rising) edge zero crossings and negative-(falling) edge zero crossings of the selected portion of the PPG signal to determine breath-time intervals caused by an influence of the respiration rate on the PPG signal; and
determining an average of the breath-time intervals for the positive-edge zero-crossings and the negative-edge zero-crossings to derive an estimate of the respiration rate.

28. The tangible computer-readable medium of claim 27, wherein the method further comprises determining a pulse rate of the subject from the PPG signal.

29. The tangible computer-readable medium of claim 27, wherein the method further comprises filtering the PPG signal with a slew-rate filter to reduce effects or eliminate signals that have slew-rates that vary more than a predetermined percentage from one pulse to the next pulse.

30. The tangible computer-readable medium of claim 29, wherein the predetermined percentage is about ±25%.

31. The tangible computer-readable medium of claim 27, wherein the sampling of the PPG signal is based on a selected frequency based on a heart rate of the subject.

32. A system to determine a respiration rate of a subject from an output of a device capable of generating a photoplethysmogram (PPG) signal, the system comprising:
one or more hardware-based processors to sample the PPG signal;
a first high-pass filter to filter the sampled PPG signal;
a second high-pass filter coupled in series with the first high-pass filter to receive an output from the first high-pass filter and to filter the output at a second selected-frequency; and
a plurality of bandpass filters to receive an output of the second high-pass filter and to determine a central frequency of various components of the PPG signal, at least one of the one or more hardware-based processors further configured to determine a spectral estimate of β, wherein β is a ratio of respiration rate to a pulse rate, from the central frequency, to determine the respiration rate.

33. The system of claim 32, further comprising:
a zero-crossing filter to receive an output from the second-high-pass filter and to interpolate positive-(rising) edge zero crossings and negative-(falling) edge zero crossings of at least a selected portion of the PPG signal to determine breath-time intervals caused by an influence of the respiration rate on the PPG signal; and
a median filter to determine an average of the breath-time intervals for the positive-edge zero-crossings and the negative-edge zero-crossings to derive an estimate of the respiration rate.

34. The system of claim 33, wherein the median filter is further configured to derive the estimate of the respiration rate based on calculating respiration rates for both the positive-edge zero crossings and the negative-edge zero crossings individually prior to averaging.

35. The system of claim 33, wherein the breath-time intervals are to be determined as a difference between interpolated zero-crossings.

36. The system of claim 32, wherein the one or more hardware-based processors are further configured to separate components of the PPG signal into a DC-modulated waveform (DC signal), an amplitude-modulated waveform (pM signal), and a frequency-modulated waveform (pT signal).

37. The system of claim 36, wherein the bandpass filters are further configured to determine a central frequency of each of the DC signal, the pM signal, and the pT signal over a range of β values.

38. The system of claim 36, further comprising a slew-rate filter to reduce effects or eliminate signals in the pM signal and the pT signal that have slew-rates that vary more than a predetermined percentage from one pulse to the next pulse.

39. The system of claim 38, wherein the predetermined percentage is about ±25%.

40. The system of claim 36, wherein the one or more hardware-based processors are to sample the PPG signal of the pM and pT signals at a selected frequency based on a heart rate of the subject.

41. The system of claim 36, wherein the one or more hardware-based processors are to sample the PPG signal of the DC signal at a selected real-time frequency.

42. The system of claim 32, wherein the one or more hardware-based processors are further configured to determine a pulse rate of the subject from the PPG signal.

43. The system of claim 32, wherein a central frequency of successive ones of the plurality of bandpass filters are based on an incremental step size of β.

44. The system of claim 43, wherein the incremental step size of β is 0.05.

45. The system of claim 44, wherein the plurality of bandpass filters comprises 13 bandpass filters.

46. The system of claim 32, wherein at least one of the first high-pass filter, the second high-pass filter, and the bandpass filters comprises a digital filter.

47. A method for determining a respiration rate of a subject from an output of a device capable of generating a photoplethysmogram (PPG) signal, the method comprising:
sampling the PPG signal at a first selected frequency;
filtering the PPG signal with a first high-pass filter;
receiving an output from the first high-pass filter and filtering the output at a second selected-frequency in a second high-pass filter;
receiving an output from the second high-pass filter and determining a central frequency of various components of the PPG signal received from the output of the second high-pass filter based on a plurality of bandpass filters; and
determining a spectral estimate of a value of β, wherein β is a ratio of respiration rate to a pulse rate, from the central frequency of the various components of the PPG signal, to determine the respiration rate.

48. The method of claim 47, further comprising:
receiving an output from the second-high-pass filter of at least a selected portion of the PPG signal and interpolating positive-(rising) edge zero crossings and negative-(falling) edge zero crossings of the selected portion to determine breath-time intervals caused by an influence of the respiration rate on the PPG signal; and
determining an average of the breath-time intervals for the positive-edge zero-crossings and the negative-edge zero-crossings to derive an estimate of the respiration rate.

49. The method of claim 48, wherein deriving the estimate of the respiration rate is based on calculating respiration rates for both the positive-edge zero crossings and the negative-edge zero crossings individually prior to averaging.

50. The method of claim 48, further comprising determining the breath-time intervals as a difference between interpolated zero-crossings.

51. The method of claim 47, further comprising separating the PPG signal into a DC-modulated waveform (DC signal), an amplitude-modulated waveform (pM signal), and a frequency-modulated waveform (pT).

52. The method of claim 51, further comprising determining a central frequency of each of the DC signal, the pM signal, and the pT signal using the plurality of bandpass filters over a range of β values.

53. The method of claim 52, further comprising, for each of the DC signal, the pM signal, and the pT signal:
calculating an average root-mean square (RMS) amplitude for signals from each bandpass filter;
normalizing the sum of the average RMS amplitude for each bandpass filter;
equalizing the average RMS amplitude values according to each of the DC signal, the pM signal, and the pT signal types; and
calculating a spectral estimate of β for each signal type.

54. The method of claim 53, further comprising calculating a merged spectrum for each of the DC signal, the pM signal, and the pT signal, the method including:
averaging the RMS amplitude values for each of the DC signal, the pM signal, and the pT signal types;
normalizing merged amplitude values; and
calculating a spectral estimate of β.

55. The method of claim 54, further comprising:
using each of a maximum value of outputs of the plurality of bandpass filters from the merged spectrum, four determined estimates of β (β_{DC}, β_{pT}, and β_{pM}, and β_{MAX, AVG}); and a four-beat average heart rate, <HR₄> as inputs to a response surface; and
determining a transfer function estimate of β (β_{XF}) from an output of the response surface.

56. The method of claim 55, further comprising:
determining zero-crossings for each of processed values of the DC signal, the pM signal, and the pT signal to determine frequency-modulation values caused by an influence of the respiration rate on the PPG signal and determine an additional β value based on the waveforms (β_{WF});
feeding back the β_{WF} value;
determining an average arithmetic value of β_{XF} and β_{WF}; and
using the arithmetic average to increase an accuracy of the respiration rate of the subject.

57. A tangible computer-readable medium having no transitory signals and containing instructions that, when executed by one or more hardware-based processors of a machine, cause the machine to perform operations comprising:
determining a respiration rate of a subject from an output of a device capable of generating a photoplethysmogram (PPG) signal, the determination including
sampling the PPG signal at a first selected frequency;
filtering the PPG signal with a first high-pass filter;
receiving an output from the first high-pass filter and filtering the output at a second selected-frequency in a second high-pass filter;
receiving an output from the second high-pass filter and determining a central frequency of various components of the PPG signal received from the output of the second high-pass filter based on a plurality of bandpass filters; and
determining a spectral estimate of a value of β, wherein β is a ratio of respiration rate to a pulse rate, from the central frequency of the various components of the PPG signal, to determine the respiration rate.

58. The tangible computer-readable medium of claim 57, wherein the method further comprises separating the PPG signal into signals including a DC-modulated waveform (DC signal), an amplitude-modulated waveform (pM signal), and a frequency-modulated waveform (pT).

59. The tangible computer-readable medium of claim 58, wherein the method further comprises determining a central frequency of each of the DC signal, the pM signal, and the pT signal using the plurality of bandpass filters over a range of β values.

60. The tangible computer-readable medium of claim 59, wherein the method further comprises, for each of the DC signal, the pM signal, and the pT signal:
calculating an average root-mean square (RMS) amplitude for signals from each bandpass filter;
normalizing the sum of the average RMS amplitude for each bandpass filter;
equalizing the average RMS amplitude values according to each of the DC signal, the pM signal, and the pT signal types; and
calculating a spectral estimate of β for each signal type.

61. The tangible computer-readable medium of claim 60, wherein the method further comprises calculating a merged spectrum for each of the DC signal, the pM signal, and the pT signal, the method including:
averaging the RMS amplitude values for each of the DC signal, the pM signal, and the pT signal types;
normalizing merged amplitude values; and
calculating a spectral estimate of β.

62. The tangible computer-readable medium of claim 61, wherein the method further comprises:
using each of a maximum value of outputs of the plurality of bandpass filters from the merged spectrum, four determined estimates of β (β_{DC}, β_{pT}, and β_{pM}, and β_{MAX, AVG}); and a four-beat average heart rate, <HR₄> as inputs to a response surface; and
determining a transfer function estimate of β (β_{XF}) from an output of the response surface.

63. The tangible computer-readable medium of claim 62, wherein the method further comprises:
determining zero-crossings for each of processed values of the DC signal, the pM signal, and the pT signal to determine an additional β value based on the zero-crossings of the waveforms (β_{WF}) in pulse time;
feeding back the β_{WF} value;
determining an average arithmetic value of β_{XF} and β_{WF}; and
using the arithmetic average to increase an accuracy of the respiration rate of the subject.
